(19) United States
(12) Patent Application Publication
(10) Pub. No.: US 2024/0366228 A1
(43) Pub. Date: Nov. 7, 2024

(54) EMBOLIC MATERIAL FOR RELIEVING OR TREATING MUSCULOSKELETAL PAIN COMPRISING FAST DISSOLVING GELATIN PARTICLES

(71) Applicant: ENGAIN CO., LTD., Gyeonggi-do (KR)

(72) Inventors: Young Gook KOH, Seongnam-si, Gyeonggi-do (KR); Jae Won SEO, Gunpo-si, Gyeonggi-do (KR); Ku Hong KWON, Seongnam-si, Gyeonggi-do (KR)

(21) Appl. No.: 18/284,374

(22) PCT Filed: Sep. 27, 2021
(86) PCT No.: PCT/KR2021/013153

§ 371 Date: Sep. 27, 2023

(30) Foreign Application Priority Data
May 17, 2021 (KR) 10-2021-0063534

(51) Int. Cl. A61B 17/12; A61K 38/39; A61L 24/10

(52) U.S. Cl. CPC A61B 17/12186; A61B 17/12109; A61K 38/39; A61L 24/104; A61L 2400/06; A61L 2430/36

(57) ABSTRACT

An embolic material for relieving or treating musculoskeletal pain comprises gelatin particles. The embolic material exhibits an effect of relieving or treating musculoskeletal pain by being injected into a blood vessel and embolized in the vessel, and then fast dissolved. Therefore, the embolic material comprising gelatin particles may be effectively used as an embolic material in the medical fields and pharmaceutical industry for relieving or treating musculoskeletal pain in patients suffering from chronic pain associated with musculoskeletal disorders.

### TECHNICAL FIELD

The present invention relates to an embolic material for relieving or treating musculoskeletal pain, and more particularly, to an embolic material for relieving or treating musculoskeletal pain comprising fast dissolving gelatin particles.

### BACKGROUND ART

Patients often seek treatment for chronic pain due to various musculoskeletal disorders. Although the pathophysiology of chronic pain has not yet been clearly established, various hypotheses have been studied and multiple treatment options have been recommended. The main purpose of treatment is to relieve pain and reduce inflammation in pain area, for example, painful joints, tendons, capsulitis around knee, shoulder, hip, elbow, wrist, ankle, finger, toe and so on. Examples of conservative treatment include nonsteroidal anti-inflammatory drugs (NSAIDs), corticosteroid agent injections, and extracorporeal shockwave therapy (ESWT). However, in some cases, recovery requires long periods of time or does not occur despite conservative treatment. When conservative treatment is ineffective, surgery is the only treatment option.

Transcatheter arterial embolization (TAE) and transcatheter arterial micro embolization (TAME) are new treatment options for patients with chronic pain that does not spontaneously improve or respond to conservative treatment. It could be considered an option before surgical treatment. Several previous studies on the mechanisms of musculoskeletal pain have suggested that abnormally developed neovessels and the accompanying nerves can be a source of pain and inflammation. Embolization of these abnormal neovessels can reduce inflammation by reducing the inflow of cells and cytokines involved in inflammation. Moreover, this embolization can reduce the growth of the accompanying sensory nerves that grow along the neovessels. Therefore, some medical centers have reported their results on TAE and TAME.

Previous studies have used imipenem-cilastatin sodium (IPM-CS) as an embolic material, and TAE using IPM-CS has resulted in high success rates. IPM-CS has been approved by the Food and Drug Administration (FDA) as an antibiotic agent because of its broad spectrum of activity against aerobic, anaerobic, Gram-positive, and Gram-negative bacteria. IPM-CS has a fast dissolution time (approximately 1 hour), and can be used as a transient embolic material during TAE. During TAE for chronic pain, IPM-CS is injected in abnormal neovessels to induce transient occlusion of target vessels. However, IPM-CS was originally created as an antibiotic; therefore, it has not been approved as an embolic material.

Conventional embolic materials, such as gelatin sponge particles and microspheres have been used for a long time and their safety and effectiveness have been sufficiently studied. The gelatin sponge used to date was a biologic substance made from purified skin gelatin. It is mainly used during vascular intervention procedures as a transient embolic material and requires about 4 weeks to dissolve. Korean Patent No. 10-1613403 discloses a method for preparing a gelatin drug delivery system, wherein the gelatin drug delivery system contains phosphate buffered saline (PBS) or phosphate buffer, and comprises a liver cancer therapeutic agent ionically bonded to gelatin.

Because those conventional gelatin particles take longer time to dissolve than IPM-CS dose, new gelatin-based embolic materials with fast-dissolving properties are needed for TAE and TAME. Therefore, it is necessary to develop new quick-soluble embolic material, which is effective in relieving or treating chronic pain associated with various musculoskeletal disorders and safe for human body, by introducing new physicochemical properties related with dissolution rate to gelatin particles for use during TAE and TAME.

### DISCLOSURE

### Technical Problem

A technical problem to be solved by the present disclosure is to provide an embolic material for relieving or treating chronic musculoskeletal pain, which is fast dissolving, effective for embolization, and safe for human body, in order to quickly relieve and treat musculoskeletal pain when applied to transcatheter arterial embolization (TAE) and transcatheter arterial micro embolization (TAME) of microvessels in patients suffering from chronic pain associated with various musculoskeletal disorders for a long time.

The technical problem to be solved by the present disclosure is not limited to the above-mentioned technical problem, and other technical problems not mentioned herein will be clearly understood by those skilled in the art to which the present invention pertains from the following description.

### Technical Solution

To solve the above technical problem, according to one aspect of the present invention, it is provided an embolic material for relieving or treating pain in musculoskeletal system, the embolic material comprising gelatin particles.

In one embodiment, the gelatin particles may have an average particle diameter of 10 to 1,000 μm.

In one embodiment, the gelatin particles may be chemically crosslinked or thermally crosslinked.

In one embodiment, the gelatin particles may be crosslinked with at least one crosslinking agent selected from the group consisting of formaldehyde, glutaraldehyde, 1-ethyl-3-(3-dimethylaminopropyl)carbodiimide (EDC), N-hydroxysuccinimide (NHS), dialdehyde starch, epoxy compounds, glutaraldehyde/glyoxal, glyoxal, 2,2-dimethoxy-2-phenylacetophenone, sodium tripolyphosphate (STPP), diacetaldehyde PEG, scleraldehyde, diethyl squarate, epichlorohydrin, genipin, tannins, phenylpropanoids, catechins, resveratrol, flavonoids, and isoflavonoids.

In one embodiment, the gelatin particles may have a solubility rate of 50% or more at 48 hours after the start of dissolution in an aqueous medium.

In one embodiment, the musculoskeletal system may be at least one selected from the group consisting of knees, shoulders, necks, elbows, wrists, ankles, fingers, and toes.

In one embodiment, the gelatin particles may be injected into a blood vessel, embolized in the vessel, thereby relieving or treating the pain in the musculoskeletal system.

In one embodiment, the gelatin particles may be injected into the blood vessel at a mean amount of 10 to 500 mg.

In one embodiment, the blood vessel may be selected from the group consisting of a femoral artery, a radial artery, a subclavian artery and a brachial artery.

In one embodiment, the pain in the musculoskeletal system may be one lasting for 6 months or more.

### Advantageous Effects

According to the present invention, it has been confirmed that, when the fast-dissolving gelatin particles are injected into the blood vessels of patients with chronic pain associated with musculoskeletal disorders during transcatheter arterial embolization (TAE) and transcatheter arterial micro embolization (TAME), the chronic musculoskeletal pain is significantly reduced without major side effects. This suggests that the fast dissolving gelatin particles of the present invention have been evaluated effective and safe for TAE and TAME with a high technical success rate to relieve or treat chronic pain.

Therefore, the embolic material comprising gelatin particles according to the present invention may be effectively used as an embolic material in the medical fields and pharmaceutical industry for relieving or treating pain related with chronic musculoskeletal disorders refractory to conservative treatment.

The effects of the present invention are not limited to the above-described effects, and it should be understood that the effects of the present invention include all effects that may be deduced from the features of the invention as described in the detailed description of the invention and the appended claims.

### DESCRIPTION OF DRAWINGS

FIG. 1 is an angiogram of the knee of a 65-year-old woman who presented with right knee pain and pain in the lateral and medial aspects of the knee joint. The woman patient was diagnosed with knee osteoarthritis with Kellgren-Lawrence classification II based on radiography of the knee. The right common femoral artery was antegrade punctured and superficial femoral artery arteriography was performed.

FIG. 2 is an angiogram of the right knee joint. Abnormal enhancement that originated from the lateral inferior genicular artery was observed in the lateral aspect of the right knee joint (arrow).

FIG. 3 is an angiogram of a blood vessel that was selected using a microcatheter and embolized using 1.2 mL of fast dissolving gelatin particles.

FIG. 4 is an angiogram showing that additional abnormal enhancement was confirmed in the medial knee joint. The descending genicular artery was selected (a) and embolized using 2.2 mL of fast dissolving gelatin particles (b).

FIG. 5 is an angiogram of blood vessels in the knee joint of a patient who has undergone embolization. During the final angiogram, no abnormal enhancement was observed in the right knee joint (arrow). The patient's baseline VAS score was 8 points, and it decreased to 2 points immediately after the procedure. The patient reported pain with a VAS score of 4 points that lasted approximately 24 hours. After that, a VAS score of 2 points was maintained until the 6-month follow-up evaluation. (VAS; visual analog scale).

FIG. 6 is a graph showing the mean VAS score change over time. All 33 patients underwent embolization had a 6-month follow-up period. The mean VAS scores at baseline, immediately after TAE, and at 1 day, 1 week, 1 month, 3 months, and 6 months after TAE were 6.67, 3.09, 4.64, 2.67, 2.30, 2.24, and 2.27, respectively (P<0.05 for baseline vs. immediately after TAE and immediately after TAE vs. 1 day after TAE). (TAE; Transcatheter arterial embolization).

FIG. 7 is a picture showing the dispersions of gelatin particles when selecting thermal crosslinking conditions.

FIG. 8 is microscopic pictures showing the dispersions of gelatin particles when selecting thermal crosslinking conditions.

### BEST MODE

The present invention provides an embolic material for relieving or treating pain in musculoskeletal system, comprising gelatin particles. Regarding pain in musculoskeletal systems, abnormally developed neovessels and the accompanying nerves in the musculoskeletal system can be a source of pain and inflammation. Thus, embolization of these abnormal neovessels can reduce inflammation by reducing the inflow of cells and cytokines involved in inflammation. The embolic material for relieving or treating musculoskeletal pain according to the present invention is an embolic material for use in a treatment procedure that controls pain by injecting gelatin particles into a blood vessel area associated with pain and forming embolization in the vessel.

The gelatin particles are made of gelatin, and the gelatin may be any material without limitation as long as it can prevent the movement of liquids such as blood flow by maintaining a solid state or a gel state for a certain period of time when coming in contact with water. Preferably, the gelatin may be mammal-derived gelatin or fish-derived gelatin. The weight-average molecular weight (Mw) of the gelatin may be 15,000 to about 400,000, preferably 30,000 to 300,000, more preferably 50,000 to 200,000, most preferably 65,000 to 100,000. The gelatin used in the present invention may be obtained from mammalian collagen or fish collagen, or may be commercially obtained, if necessary.

In the present invention, it has been found that gelatin particles having a specific particle size, among gelatin particles used in conventional embolization, are useful for peripheral blood vessels in a specific musculoskeletal system when used in the embolization procedure, inducing embolization and then dissolution in a short time, showing that embolization using the above gelatin particles effectively relieves a long-term incurable musculoskeletal pain. For this purpose, the gelatin particles that are used in the present invention have a size that may be solidified or gelled and dissolve within a short time after being injected into microvessels of joints or the like. Preferably, the gelatin particles that are used in the present invention may have an average particle diameter of 10 to 1000 μm, more preferably 20 to 800 μm, 30 to 500 μm, 40 to 300 μm, or 50 to 150 μm, most preferably 80 to 100 μm.

In one embodiment, the gelatin particles may be crosslinked using a crosslinking agent, and the crosslinking may be chemical crosslinking or thermal crosslinking. A crosslinking agent that is used for chemical crosslinking of the gelatin particles may be, but not limited to, at least one selected from among formaldehyde, glutaraldehyde, 1-ethyl-3-(3-dimethylaminopropyl)carbodiimide (EDC, which is used alone or together with N-hydroxysuccinimide (NHS)), dialdehyde starch, epoxy compounds, glutaraldehyde/glyoxal, glyoxal, 2,2-dimethoxy-2-phenylacetophenone, sodium tripolyphosphate (STPP), diacetaldehyde, scleraldehyde, diethyl squarate, epichlorohydrin, genipin, tannins, phenylpropanoids, catechins, resveratrol, flavonoids (quercetin, etc.), and isoflavonoids (isoflavone, etc.). Thermal crosslinking of the gelatin particles may be performed at a temperature of 120° C. or above, preferably 130° C. or above.

In the present invention, fast dissolution and quick-soluble property of the gelatin particles may reduce the incidence and duration of ischemic pain. For this purpose, the solubility rate of the gelatin particles in an aqueous medium may be 50% or more at 48 hours after the start of dissolution; 80% or more at 48 hours; more preferably 98% or more at 48 hours; further more preferably 92% or more at 24 hours, 98% or more at 48 hours; most preferably 87% or more at 30 minutes, 89% or more at 3 hours, 92% or more at 24 hours, 98% or more at 48 hours after the start of dissolution. The expression “solubility rate of the gelatin particles in an aqueous medium” may refer to the solubility rate of the gelatin particles in an aqueous medium such as water, normal saline, blood, serum, or other aqueous media that may be used in the human body.

The embolic material of the present invention is intended to relieve or treat pain that has occurred in musculoskeletal system, wherein the musculoskeletal system is at least one selected from the group consisting of knees, shoulders, necks, elbows, wrists, ankles, fingers and toes, but not limited thereto.

In one embodiment, the gelatin particles may be injected into a blood vessel, embolized in the vessel, thereby relieving or treating the pain in the musculoskeletal region. For this purpose, the gelatin particles may be injected into the blood vessel preferably at a mean amount of 10 to 500 mg, more preferably 50 to 400 mg, most preferably 100 to 300 mg. The blood vessel refers to the component of blood circulation system including vein, artery, capillary and so on. The blood vessel may be selected from the group consisting of a femoral artery, a radial artery, a subclavian artery, and a brachial artery, but not limited thereto.

IPM-CS has been used as embolic materials in TAE related with musculoskeletal disorder pain, and microspheres has been used as embolic materials in conventional TAE. When mixed with a contrast agent, IPM-CS forms crystalline particles that can induce transient embolic effect. IPM-CS almost completely dissolves within 24 hours after the transient embolized effects on the vessels, but it has not been approved as an embolic material because it was originally created as an antibiotic. Unlike IPM-CS, gelatin particles have been used for a long time as an embolic material for vascular embolization, therefore, the convenience, usefulness and stability of the gelatin particles have been sufficiently recognized. Accordingly, gelatin particles are used for transcatheter arterial embolization (TAE) and transcatheter arterial micro embolization (TAME) in the present invention, but faster dissolving gelatin particles are used to obtain similar effect to IPM-CS since conventionally used gelatin particles have a longer dissolution time than IPM-CS (usually 7 to 28 days). That is, novel fast-dissolving gelatin particles that are used in one embodiment of the present invention are particles for use in transcatheter arterial micro-embolization (TAME), also referred to as embolization for musculoskeletal disorder pain. The fast dissolving gelatin particles that are used in one embodiment of the present invention have a particle size of 50 to 150 μm (average 90 μm), and the solubility rates thereof are 87% at 30 minutes, 89% at 3 hours, 92% at 24 hours, and 98% at 48 hours. In an Example of the present invention, the technical success rate was 100% and the clinical success rate was 75.76% at 6 months after the procedure. This clinical success rate is similar to the average rate reported by previous studies that used IPM-CS as an embolic material.

According to the present invention, it is possible to relieve or treat pain in musculoskeletal system by injecting the gelatin particles into a blood vessel, the gelatin particles being embolized in the vessel, thereby reducing the growth of accompanying sensory nerves that grow along the neovessels in a painful area.

In one embodiment, the pain in the musculoskeletal system may be chronic pain, particularly chronic pain lasting for 6 months or more, but not limited thereto. The effectiveness of the embolic material comprising gelatin particles according to the present invention for relieving or treating pain in musculoskeletal system was confirmed by injecting the embolic material to patients who did not experience improvements in pain with various conservative treatments, reported pain lasting more than 6 months, and wanted other treatments that could be performed before having to resort to surgery.

### MODE FOR INVENTION

Hereinafter, the present invention will be described in more detail with reference to examples. However, the following examples are intended to illustrate the present invention, and the scope of the present invention is not limited thereto.

### Example 1. Preparation of Gelatin Particles

### (1) Chemical Crosslinking

### [Preparation of Formaldehyde Solution (FA)]

1) 50 mL of distilled water was placed in a 50 mL centrifuge tube with a micropipette.
2) 88 μL of the distilled water was removed, and 88 μL of a 36-38% formaldehyde solution was added.
3) The solution was used in portions of 11.75 mL.

### [Preparation of Gelatin Particles (IPZA)]

1) Gelatin was dissolved in distilled water (7 g of gelatin, and 100 mL of distilled water).
The volume of distilled water was measured using a mass cylinder.
The temperature was checked (50° C.).
2) The gelatin solution was transferred into a 1 L beaker and the temperature was observed to decrease.
The temperature was checked (40° C.).
3) When the temperature of the gelatin solution reached 40° C., the FA solution prepared in advance was added.
4) The resulting solution was stirred using a stirrer (most strongly for 7 min).
5) The resulting gelatin foam was transferred onto a plate, and then immediately placed in a freezer (at −50° C. for 40 hours).
6) The frozen gelatin foam was freeze-dried.
7) The freeze-dried gelatin foam was pulverized into particles.
8) The gelatin particles were sieved using a sieve.

### (2) Preparation of Gelatin Particles by Thermal Crosslinking

1) 7 g of gelatin (160 to 175 bloom) was added to 100 mL of distilled water and dissolved with being stirred on a hot plate.
2) When the gelatin was completely dissolved, the gelatin solution was transferred into a beaker (1 L) and allowed to stand until it reached 40° C.
3) The gelatin solution was whipped at 40° C. for 7 minutes.
4) The gelatin foam was transferred onto a plate and frozen (1 day).
5) Freeze drying was performed (3 days).
6) The freeze-dried gelatin sponge particles (GSPs) were taken out and placed in a beaker, and the opening of the beaker was sealed using KIMTECH to prevent dust from entering.
7) The temperature of an oven was adjusted to 135° C., and when the temperature became constant (after about 2 hours and 30 minutes), the GSPs in the beaker were quickly put in the oven.
8) The vacuum pump was operated for 24 hours from the time switched on.
9) The GSPs were taken out and cooled at room temperature.
10) Pulverization was carried out.
11) The particles were sieved using a sieving machine. (If particles remaining after sieving are collected and sieved again, the yield increases).
12) Blister packaging was carried out.
13) Gamma ray sterilization was carried out.

### Example 2. Selection of Thermal Crosslinking Conditions

Thermal crosslinking temperature was set to 135° C. according to the following tests, in which dispersion stability test and microscopic observation of gelatin particles were performed.

### Dispersion Stability Test

To prepare gelatin particles that can be stably dispersed without dissolving when mixed with ‘normal saline (physiological saline)+contrast agent’ for embolization, the temperature and time at which gelatin particles are prepared by thermal crosslinking were evaluated. The results are shown in Table 1 below.

TABLE 1

| Temperature | Time (hrs) | | | | |
|---|---|---|---|---|---|
| (° C.) | 8 | 16 | 24 | 32 | 48 |
| 120 | X | X | X | X | X |
| 125 | X | X | X | X | X |
| 130 | X | X | Δ | Δ | — |
| 135 | — | — | ◯ | — | — |

X: unstable,
Δ: slightly stable,
◯: stable

### (2) Microscopic Observation of Gelatin Particles

Gelatin particles (Lot: 190523) were dissolved in a mixture solution of physiological saline and contrast (1:1) at a concentration of 10 mg/mL, and the dispersion was observed visually and under a microscope. Conditions for preparation of the gelatin particles are shown in Table 2 below.

TABLE 2

| Sample No. | Conditions |
|---|---|
| 1 | 125° C. and 32 hrs |
| 2 | 130° C. and 24 hrs |
| 3 | 130° C. and 32 hrs |
| 4 | 135° C. and 24 hrs |
| 5 | EGgel-SS* |

*Embolization product used as a control, which is used in liver cancer embolization.

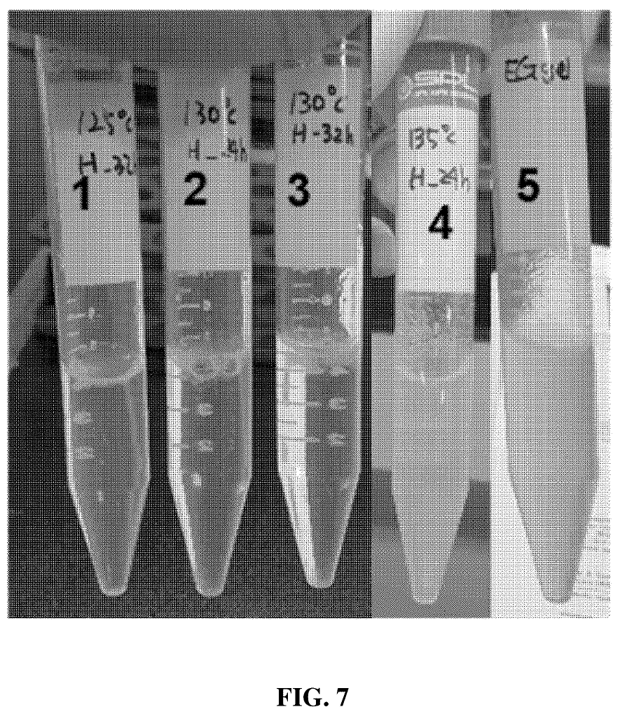

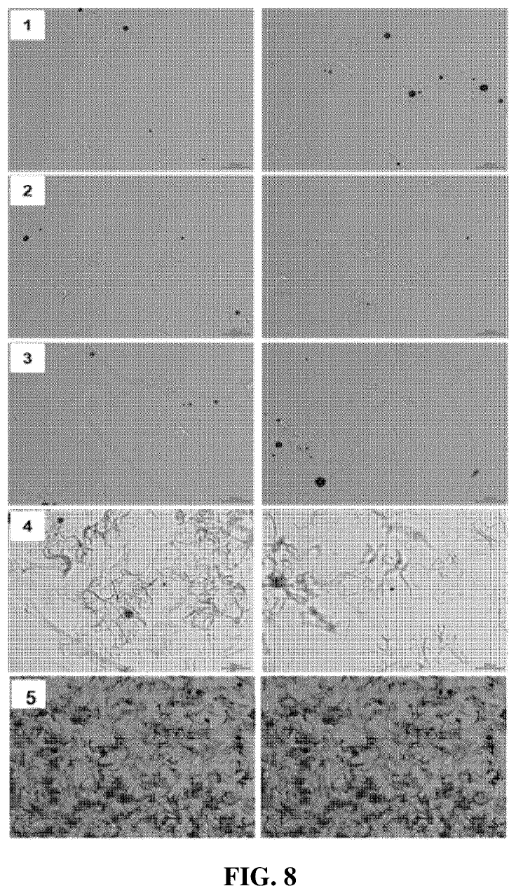

FIG. 7 shows the results of visually observing the gelatin particle dispersions, and FIG. 8 shows the results of observing the gelatin particle dispersions under a microscope.

FIG. 7 shows the result of comparative evaluation of the dissolution patterns of the samples (Nos. 1, 2, 3 and 4) prepared by the thermal crosslinking method and the commercially available embolization product ‘EGgel S PLUS’ (Engain Co., Ltd., Korea) (No. 5). It was found that sample Nos. 1, 2 and 3 were observed visually to be dissolved, not dispersed, when mixed with the mixture solution (physiological saline and contrast agent). On the contrary, sample Nos. 4 and 5 were dispersed, not dissolved, while maintaining their sponge structure when mixed with the mixture solution.

In addition, FIG. 8 shows the result of microscopic state when each of the samples (No. 1, 2, 3, 4 and 5) mixed with the mixture solution (physiological saline and contrast agent). It was found that each of sample Nos. 1, 2 and 3 was mostly dissolved when mixed with the mixture solution (physiological saline and contrast agent), but sample Nos. 4 and 5 were dispersed, not dissolved, while maintaining their sponge structure when mixed with the mixture solution.

### Example 3. Embolization Procedure

### (1) Patients

In a study conducted in this Example, TAE was performed on patients with musculoskeletal disorders. Patients included in this study did not experience improvements in pain with various conservative treatments, reported pain lasting more than 6 months, and wanted other treatments that could be performed before having to resort to surgery.

This retrospective study was performed at a tertiary care center after receiving institutional review board approval. All patients provided informed consent to undergo TAE as an alternative treatment.

All patients who underwent the procedure were adults 18 years or older and had chronic pain (over than 5 points of VAS) due to musculoskeletal disorders for more than 6 months. Symptoms and the results of imaging tests, such as radiography, ultrasound, or magnetic resonance imaging, were used to diagnose musculoskeletal disorders.

Diagnoses included knee osteoarthritis (OA), lateral and medial epicondylitis, and adhesive capsulitis. Patients reported chronic pain that did not respond to conservative treatments (over than 6 months) such as medication (NSAIDs), corticosteroid injections, physical therapy, and ESWT; therefore, they requested TAE to relieve pain. The exclusion criteria were age younger than 18 years and infection at the site of pain. Patient selection was performed using a multidisciplinary approach in cooperation with interventional radiologists and orthopedic surgeons.

This study enrolled 29 patients (33 cases) from August 2019 to January 2020. Procedures were performed to improve chronic knee pain (23 cases of OA), elbow pain (4 cases of lateral and 5 cases of medial epicondylitis), and shoulder pain (1 case of adhesive capsulitis). There were 16 male and 17 female patients with a mean age of 60±10.9 years (range, 37 to 80 years). The mean duration of symptoms was 29.6.±17.4 months (range, 6 to 60 months; median, 30 months). Conservative treatments that were previously used included the administration of pain relievers (nonsteroidal anti-inflammatory drugs), physical therapy, corticosteroid injections, and ESWT. All patients with knee pain had mild to moderate OA with Kellgren-Lawrence grade 2 or 3 according to radiography or magnetic resonance imaging.

### (2) Embolization Procedure

The arterial access was obtained via the common femoral artery (CFA) or radial artery. Under local anesthesia, a percutaneous arterial approach was performed using a 5-Fr introducer sheath (Terumo, Tokyo, Japan) via the CFA (28 cases) or a 4-Fr sheath (Terumo) via the radial artery (5 cases).

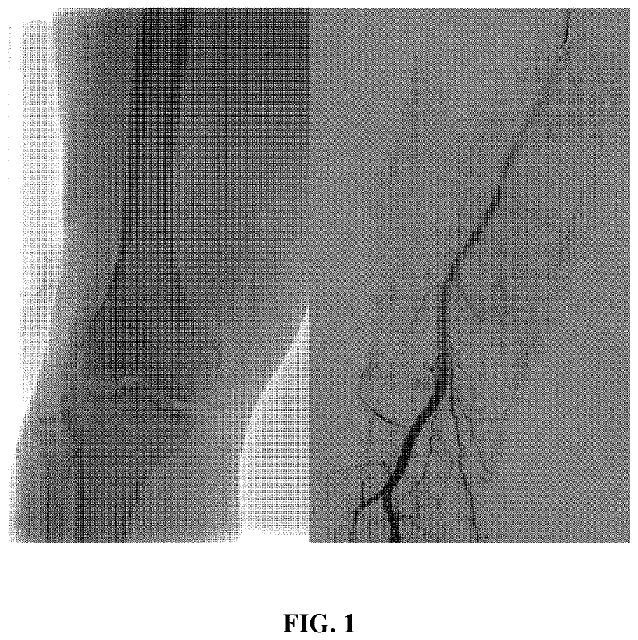

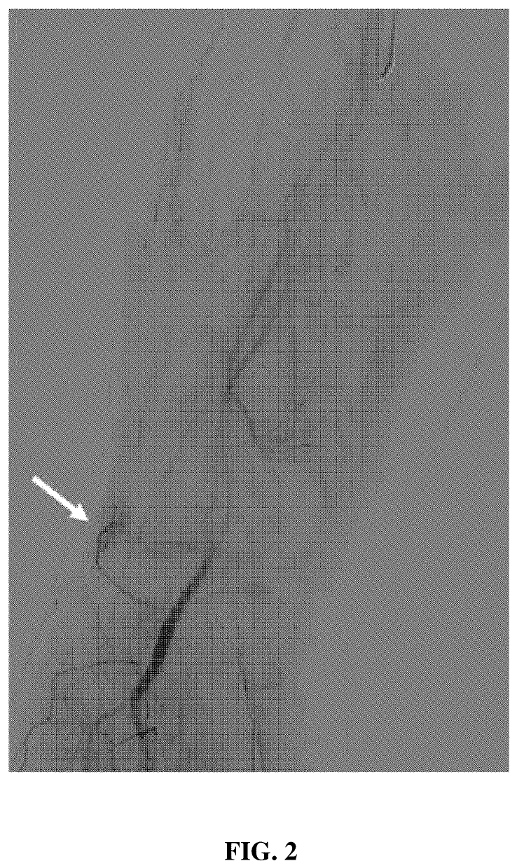

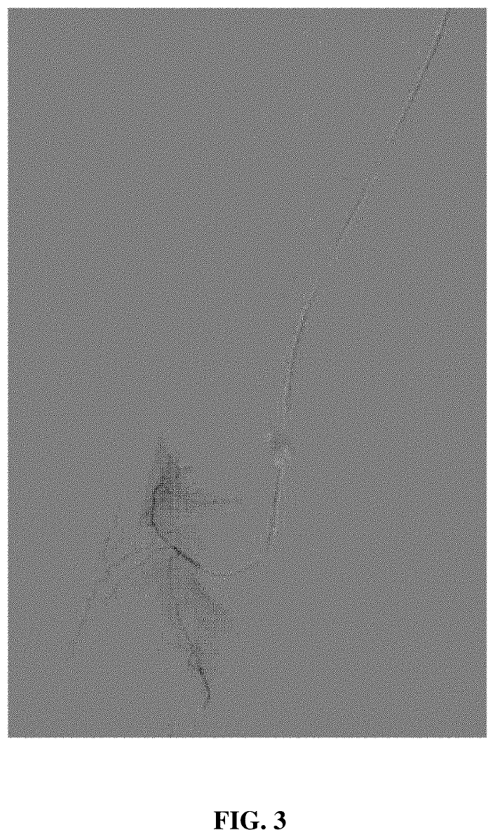

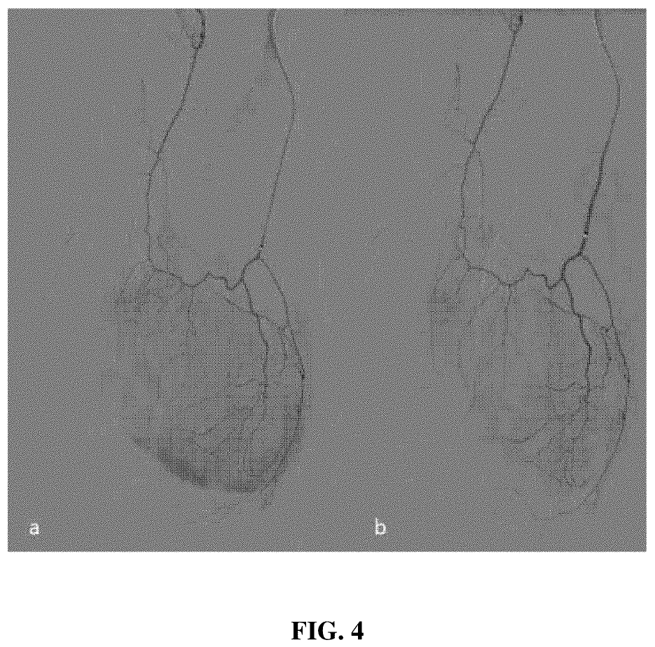

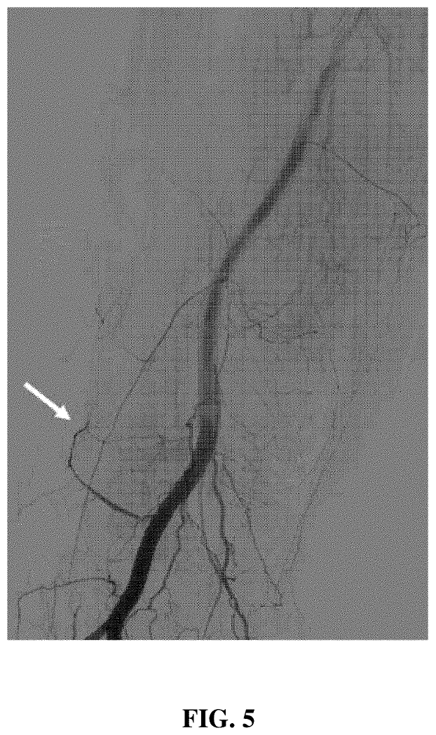

Baseline arteriography was performed using a 4-Fr or 5-Fr angiographic catheter (Glidecath, non-taper angle; Terumo) via the superficial femoral artery of patients with knee pain (FIG. 1) and via the subclavian and brachial arteries of patients with shoulder or elbow pain. Arteriography detected abnormal enhancement of neovessels in the painful area (FIG. 2, arrow). The corresponding artery of the target site was superselected using a coaxial 1.9-Fr microcatheter (Tellus; Asahi Intecc, Nagoya, Japan) and a 0.016-inch microguidewire (Meister; Asahi Intecc). Through this microcatheter, quick-soluble gelatin particles were injected without reflux until the flow of the selected artery was significantly slowed and stasis was nearly achieved (FIGS. 3 and 4a). Angiography was performed again to confirm that abnormal staining was no longer clearly visible (FIGS. 4b and 5). After completion of the procedure, hemostasis of the puncture site was achieved using a closure device (Mynx; Cordis Corporation, NJ, USA) for the CFA or radial compression device (PreludeSYNC; Merit Medical Systems, South Jordan, Utah, USA) for the radial artery. Patients who achieved CFA hemostasis with a closure device were able to ambulate after 4 hours of absolute bed rest and confirmation that there were no complications of the puncture site. In the case of the radial artery, patients rested for 2 hours with a radial compression device applied to the wrist. The patients were discharged on the day of the procedure or after one day of hospitalization as they wanted.

Fast dissolving (Quick-soluble) gelatin particles (IPZA; Engain, Gyeonggi-do, Republic of Korea) with a diameter of 50 to 150 μm (average, 90 μm) were used for all cases. According to the physical property tests, the solubility rates of these fast dissolving gelatin particles in normal saline were 87% at 30 minutes, 89% at 3 hours, 92% at 24 hours, and 98% at 48 hours. Fast dissolving gelatin particles were mixed with normal saline and iodine contrast agent (Visipaque; GE Healthcare, Little Chalfont, United Kingdom) via a pumping method with multiple passages so they could be visualized during fluoroscopic examination and delivery.

### (3) Assessment and Follow-up

Clinical information and imaging data of patients and procedures reported in the electronic records and picture archiving and communication system were reviewed. Technical success was defined as selective embolization of at least one feeding artery to the painful area.

The degree of pain was recorded using a 10-point visual analog scale (VAS), with 0 indicating no pain and 10 indicating the maximum pain intensity. The VAS scores were assessed at baseline, immediately after the procedure, and at 1 day, 1 week, 1 month, 3 months, and 6 months after the procedure. Clinical success was defined as a decrease in the VAS score of more than 50% from that at baseline. Adverse events were assessed during or after the procedure and included changes in skin color of the treated area, puncture site hematoma, muscle weakness, parasthesia, and allergic reactions to iodine contrast agent; these were investigated and recorded.

### (4) Statistical Analysis

Categorical variables are expressed as percentages and continuous variables are expressed as means and standard deviations. The Wilcoxon signed-rank test was used to compare baseline and sequential follow-up VAS scores. Student's t-test was used to analyze the change in the VAS score according to the time after the procedure. P<0.05 was considered statistically significant. All statistical analyses were performed using SPSS software (version 17.0; SPSS, Chicago, IL, USA).

### (5) Results

The technical success rate was 100% ( 33/33). The fast dissolving gelatin particles were used for all procedures, and the mean amount of embolic agent used was 4.3±1.9 mL (based on 100 mg gelatin particles: 2 mL physiological saline: 8 mL contrast agent). The average number of arteries per embolization was 1.8±0.8.

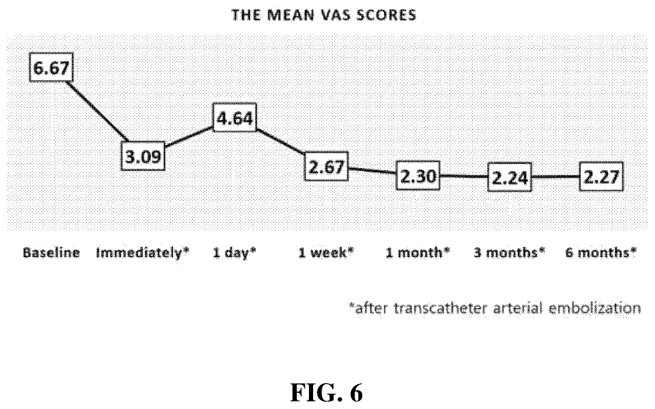

The follow-up duration was 6 months for all 33 patients. The mean VAS scores at baseline, immediately after TAE, and at 1 day, 1 week, 1 month, 3 months, and 6 months after TAE were 6.67, 3.09, 4.64, 2.67, 2.30, 2.24, and 2.27, respectively (P<0.05 for baseline vs. immediately after TAE and immediately after TAE vs. 1 day after TAE, FIG. 6). The clinical success rate of the VAS score being reduced by less than half was 75.76% ( 24/33) at 6 months after the procedure. The overall mean pain score gradually decreased during the follow-up period. However, in 18 of the 33 cases, the pain became worse after hours of procedure than immediately after the procedure. The pain persisted for an average of 24 to 48 hours and then improved. Patients who participated in the study had a mean pretreatment baseline VAS score of 6.67. The average VAS score decreased to 3.09 points immediately after the procedure, but it increased significantly to 4.64 points the day after the procedure (FIG. 6). Pain medications were administered to control pain, and the pain improved before the 1-week follow-up examination. The post-procedural pain locations were all correlated with the area where the embolized vessels are distributed. Therefore it was thought to be transient ischemic pain caused by embolization. It is thought that faster dissolution of the gelatin particles could reduce the incidence and duration of ischemic pain. In addition, the small size of gelatin particles could be embolized far peripheral branches, inducing ischemic pain.

No major adverse events were reported after the embolization procedure. Transient erythematous skin reactions in the embolized area occurred in 22 cases (66.7%), and hematoma at the puncture site (inguinal area over the CFA) occurred in 3 cases. Mild allergic reactions (skin rash and itching) to the iodine contrast agent were observed in one case. There have been no reports of any new muscle weakness or paresthesia.

The above description of the present invention is exemplary, and those of ordinary skill in the art will appreciate that the present invention can be easily modified into other specific forms without departing from the technical spirit or essential characteristics of the present invention. Therefore, it should be understood that the embodiments described above are exemplary in all aspects and are not restrictive. For example, each component described to be of a single type can be implemented in a distributed manner. Likewise, components described to be distributed can be implemented in a combined manner.

The scope of the present invention is defined by the following claims. It shall be understood that all modifications and embodiments conceived from the meaning and scope of the claims and equivalents thereto are included within the scope of the present invention.

1. A method for relieving or treating pain in musculoskeletal system, comprising step of:
injecting, to a subject, an embolic material comprising gelatin particles.

2. The method of claim 1, wherein the gelatin particles have an average particle diameter of 10 to 1,000 μm.

3. The method of claim 1, wherein the gelatin particles are chemically crosslinked or thermally crosslinked.

4. The method of claim 1, wherein the gelatin particles are crosslinked with at least one crosslinking agent selected from the group consisting of formaldehyde, glutaraldehyde, 1-ethyl-3-(3-dimethylaminopropyl)carbodiimide (EDC), N-hydroxysuccinimide (NHS), dialdehyde starch, epoxy compounds, glutaraldehyde/glyoxal, glyoxal, 2,2-dimethoxy-2-phenylacetophenone, sodium tripolyphosphate (STPP), diacetaldehyde PEG, scleraldehyde, diethyl squarate, epichlorohydrin, genipin, tannins, phenylpropanoids, catechins, resveratrol, flavonoids, and isoflavonoids.

5. The method of claim 1, wherein the gelatin particles have a solubility rate of 50% or more at 48 hours after the start of dissolution in an aqueous medium.

6. The method of claim 1, wherein the musculoskeletal system is at least one selected from the group consisting of knees, shoulders, necks, elbows, wrists, ankles, fingers, and toes.

7. The method of claim 1, wherein the gelatin particles are injected into a blood vessel, embolized in the vessel, thereby relieving or treating the pain in the musculoskeletal system.

8. The method of claim 7, wherein the gelatin particles are injected into the blood vessel at a mean amount of 10 to 500 mg.

9. The method of claim 7, wherein the blood vessel is one selected from the group consisting of a femoral artery, a radial artery, a subclavian artery and a brachial artery.

10. The method of claim 1, wherein the pain in the musculoskeletal system is one lasting for 6 months or more.
